(19) United States
(12) Patent Application Publication
(10) Pub. No.: US 2018/0199916 A1
(43) Pub. Date: Jul. 19, 2018

(54) DIAGNOSTIC IMAGING CATHETER

(71) Applicant: TERUMO KABUSHIKI KAISHA, Tokyo (JP)

(72) Inventors: Soichiro SUGIHARA, Fujinomiya-shi (JP); Yuuki SAKAGUCHI, Fujinomiya-shi (JP)

(73) Assignee: TERUMO KABUSHIKI KAISHA, Tokyo (JP)

(21) Appl. No.: 15/921,330

(22) Filed: Mar. 14, 2018

Related U.S. Application Data
(63) Continuation of application No. PCT/JP2016/074393, filed on Aug. 22, 2016.

(30) Foreign Application Priority Data
Sep. 18, 2015 (JP) 2015-186014

(51) Int. Cl. A61B 8/00; A61B 5/00; A61B 8/12

(52) U.S. Cl. CPC A61B 8/445; A61B 5/0066; A61B 5/6852; A61B 8/12; A61B 5/0084

(57) ABSTRACT

A sheath of a catheter for image diagnosis includes a first filter placed in a first communication hole, allowing a flow of gas to an outside from an inside of a lumen via the first communication hole, and restricting a flow of a component scattering or absorbing light contained in a body fluid to the inside from the outside of the lumen and a second filter placed in a second communication hole, allowing a flow of a liquid component contained in the body fluid to the inside from the outside of the lumen via the second communication hole, and restricting a flow of the component scattering or absorbing the light to the inside from the outside of the lumen.

### CROSS-REFERENCES TO RELATED APPLICATIONS

This application is a continuation of International Application No. PCT/JP2016/074393 filed on Aug. 22, 2016, which claims priority to Japanese Application No. 2015-186014, filed on Sep. 18, 2015, the entire content of both being incorporated herein by reference.

### TECHNICAL FIELD

The present invention relates to a catheter for image diagnosis.

### BACKGROUND ART

In the related art, for image diagnosis using intravascular ultrasound (IVUS), optical coherence tomography (OCT), or optical frequency domain imaging (OFDI), a catheter is used for acquisition of a diagnostic image for performing diagnosis on a disease site in a living body and the like.

In addition, in recent years, a catheter for image diagnosis in which an IVUS function is combined with OCT and OFDI functions has been proposed as inJP-T-2010-508973. The catheter for image diagnosis is provided with a drive shaft in which an ultrasound transferring and receiving unit transferring and receiving an ultrasound wave and an optical transceiver transferring and receiving light are disposed and a sheath provided with a lumen into which the drive shaft is inserted to be capable of moving forward and backward. With the catheter for image diagnosis, two types of tomographic images can be obtained that utilize the IVUS characteristics to be capable of measuring up to a high depth region and the OCT characteristics to be capable of measuring with high resolution.

When the catheter for image diagnosis is used, a priming treatment for filling the sheath with a priming solution such as a saline solution is performed so that the ultrasound wave is efficiently transferred and received. In addition, since the light is scattered or absorbed by a component scattering or absorbing the light that is present in blood, such as an erythrocyte, the blood in the vicinity of a part observed with the catheter for image diagnosis needs to be temporarily removed in a case where the inside of a blood vessel is observed by OCT or OFDI. Accordingly, in general, a so-called flush treatment for blood removal by a flush solution containing a contrast agent or the like is performed.

In general, a communication hole for communication between the inside and the outside of the lumen is disposed in the sheath of the catheter for image diagnosis and the catheter for image diagnosis is configured such that the priming solution with which the sheath is filled during the priming treatment is discharged to the outside of the sheath via the communication hole along with the air in the sheath.

However, the catheter for image diagnosis has a negative internal pressure during a so-called pull-back operation in which the drive shaft is moved backward while the catheter for image diagnosis is used, and thus blood may flow into the sheath into which the optical transceiver is inserted via the communication hole disposed in the sheath. When blood is present around the optical transceiver, the diagnostic image acquired by the optical transceiver may become unclear due to the erythrocyte.

In addition, since the flush treatment is to temporarily remove blood, the flush solution supplied into the blood vessel may be caused to flow by the blood flow and the distal side of the sheath may be covered again with the blood once a certain period of time elapses after the flush treatment is performed, and then the diagnostic image acquired by the optical transceiver may become unclear. Although it is conceivable as an example to perform an operation such that flush completion and pull-back initiation timings coincide with each other, so that the pull-back operation is completed before blood inflow around the optical transceiver, it is difficult to make the timings of the operations coincide with each other in view of the blood flow and a smooth progress of procedure can be hindered. Also conceivable is to lengthen the time (ischemic time) in which the flush solution stays around the optical transceiver by increasing the amount of the flush solution that is sent into the blood vessel. However, once the flush solution containing the contrast agent is sent in quantity into the blood vessel, complications such as nephropathy may occur and a patient's burden may increase.

### SUMMARY

The disclosure herein provides a catheter for image diagnosis capable of inhibiting infiltration of a component scattering or absorbing light contained in a body fluid into a sheath and reducing the burden on a patient by reducing the amount of a flush solution used for a flush treatment.

according to one aspect of the disclosure, a catheter for image diagnosis includes a drive shaft including a distal portion where an ultrasound transferring and receiving unit transferring and receiving an ultrasound wave and an optical transceiver transferring and receiving light are placed and a sheath including a lumen into which the drive shaft is inserted to be capable of moving forward and backward. The sheath includes a first communication hole formed in a distal portion and allowing an inside and an outside of the lumen to communicate with each other, a second communication hole formed on a side closer to a proximal side than the first communication hole and allowing the inside and the outside of the lumen to communicate with each other, a first restriction unit placed in the first communication hole, allowing a flow of gas to the outside from the inside of the lumen via the first communication hole, and restricting a flow of a component scattering or absorbing light contained in a body fluid to the inside from the outside of the lumen, and a second restriction unit placed in the second communication hole, allowing a flow of a liquid component contained in the body fluid to the inside from the outside of the lumen via the second communication hole, and restricting a flow of the component scattering or absorbing the light contained in the body fluid to the inside from the outside of the lumen.

In a further aspect of the disclosure, with the catheter for image diagnosis configured as described above, the air in the lumen can be discharged from the first communication hole and the second communication hole during a priming treatment, and thus the air in the lumen can be replaced with a priming solution up to a distal side. In addition, during a pull-back operation, the first restriction unit and the second restriction unit are capable of restricting a flow of the component scattering or absorbing the light contained in the body fluid to the inside from the outside of the lumen via the first communication hole and the second communication hole. As a result, a diagnostic image becoming unclear due to the component scattering or absorbing the light can be inhibited. Furthermore, the negative pressure in the lumen can be eliminated by a flow of the liquid component, contained in the body fluid flowing in from the proximal side, to the inside of the lumen via the second communication hole. Moreover, the amount of the body fluid that flows in from the proximal side in the body lumen and flows into the side which is closer to the distal side than the second communication hole can be decreased by the body fluid flowing in from the proximal side in the body lumen being guided to the second communication hole. As a result, the region where the body fluid is swept away by a flush treatment can be narrowed, and thus the amount of a flush solution used for the flush treatment can be reduced and the burden on a patient can be reduced.

### BRIEF DESCRIPTION OF DRAWINGS

FIG. 1 is a plan view illustrating a state where an external device is connected to a catheter for image diagnosis according to an exemplary embodiment of the disclosure.

FIG. 2(A) is a side view of the catheter for image diagnosis according to the exemplary embodiment of the disclosure on which a pull-back operation (crafting operation) is yet to be performed, and FIG. 2(B) is a side view of the catheter for image diagnosis at a time when the pull-back operation is performed.

FIG. 3 is an enlarged sectional view illustrating a configuration of a distal side of the catheter for image diagnosis according to the exemplary embodiment of the disclosure.

FIG. 4 is an enlarged sectional view illustrating a configuration of a proximal side of the catheter for image diagnosis according to the exemplary embodiment of the disclosure.

FIG. 5(A) is a sectional view illustrating how a priming treatment is performed with the catheter for image diagnosis according to the exemplary embodiment of the disclosure, and FIG. 5(B) is a sectional view illustrating a state where the catheter for image diagnosis is inserted into a blood vessel, and FIG. 5(C) is a sectional view illustrating how a flush treatment is performed.

FIG. 6(A) is a sectional view illustrating how the pull-back operation is performed with the catheter for image diagnosis according to the exemplary embodiment of the disclosure, and FIG. 6(B) is a sectional view illustrating how the pull-back operation is completed.

### DESCRIPTION

Hereinafter, an exemplary embodiment of the disclosure will be described with reference to the accompanying drawings. Note that the following description does not limit the technical scope and the meanings of the terms that are described in the claims. In addition, the dimension ratios in the respective drawings are exaggerated for convenience of description and differ from the actual ratios in some cases.

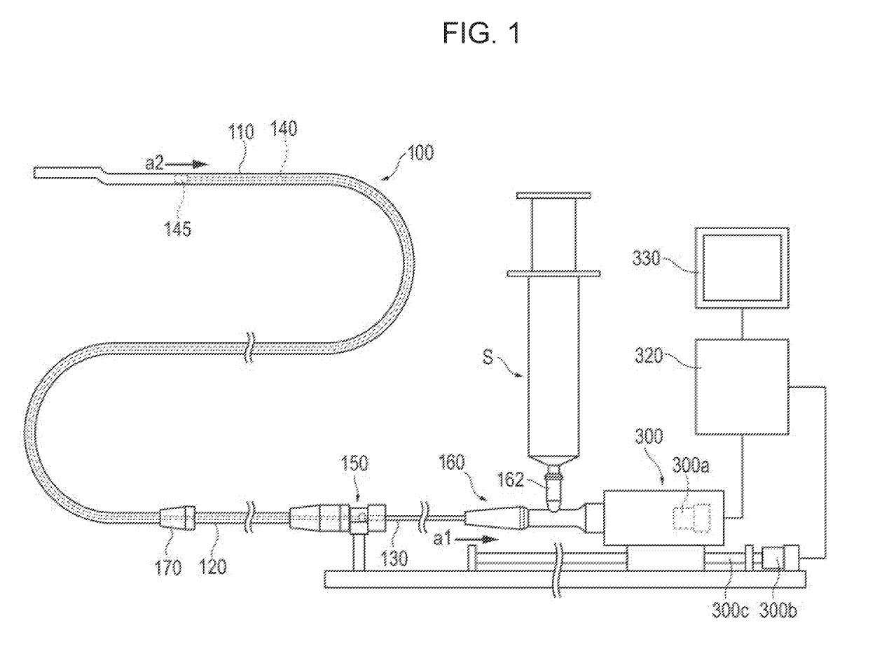

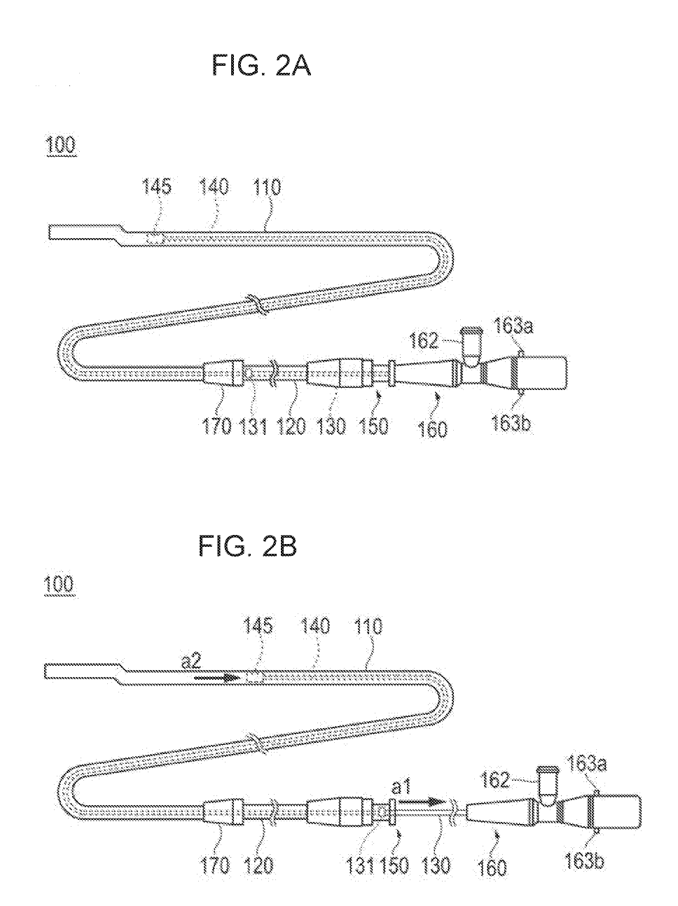

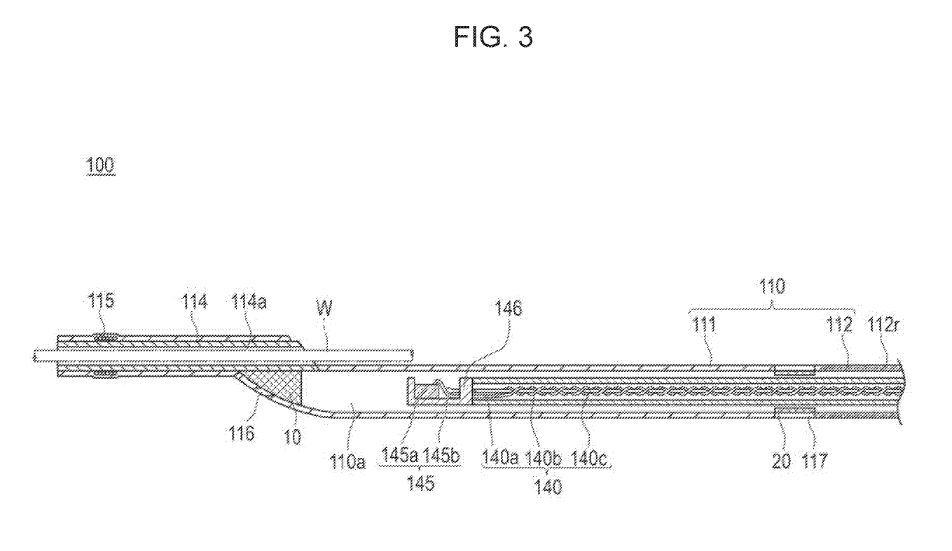

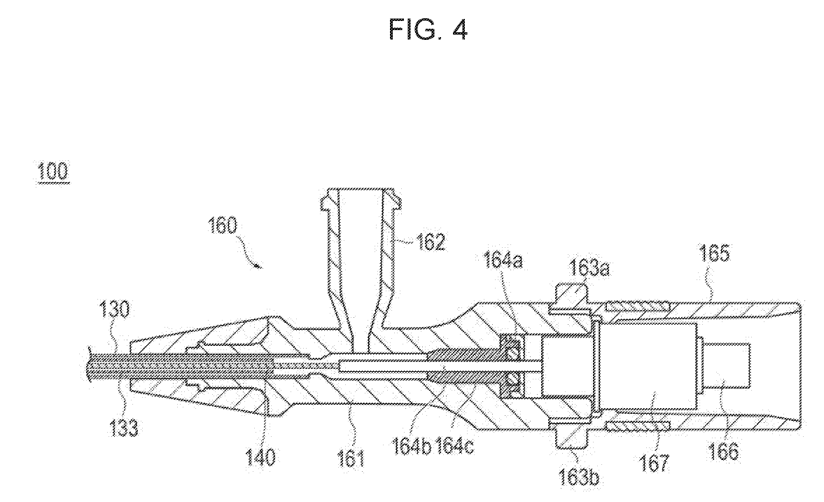

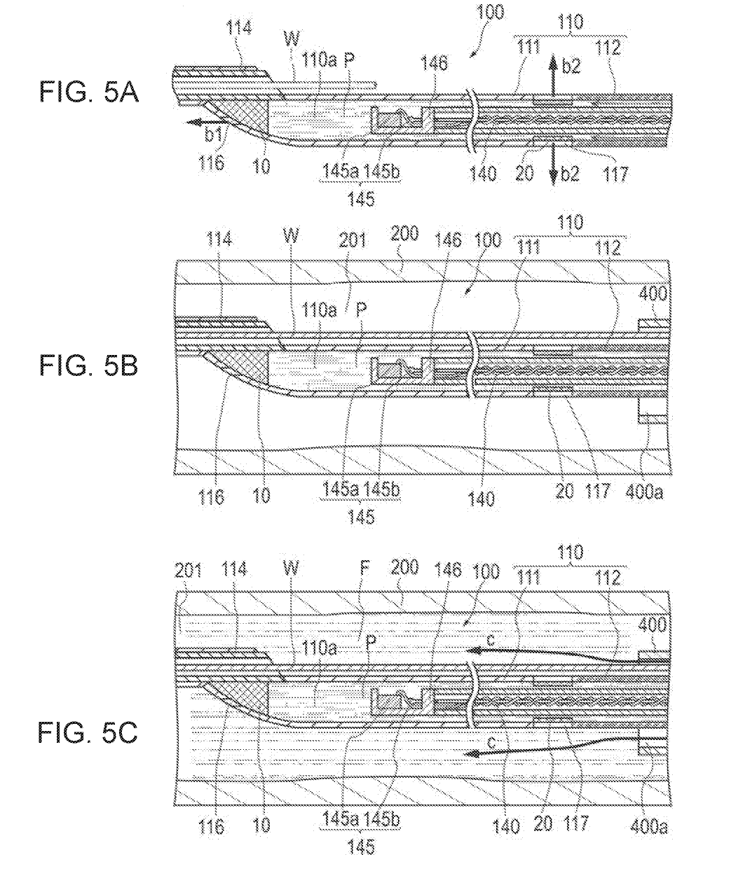

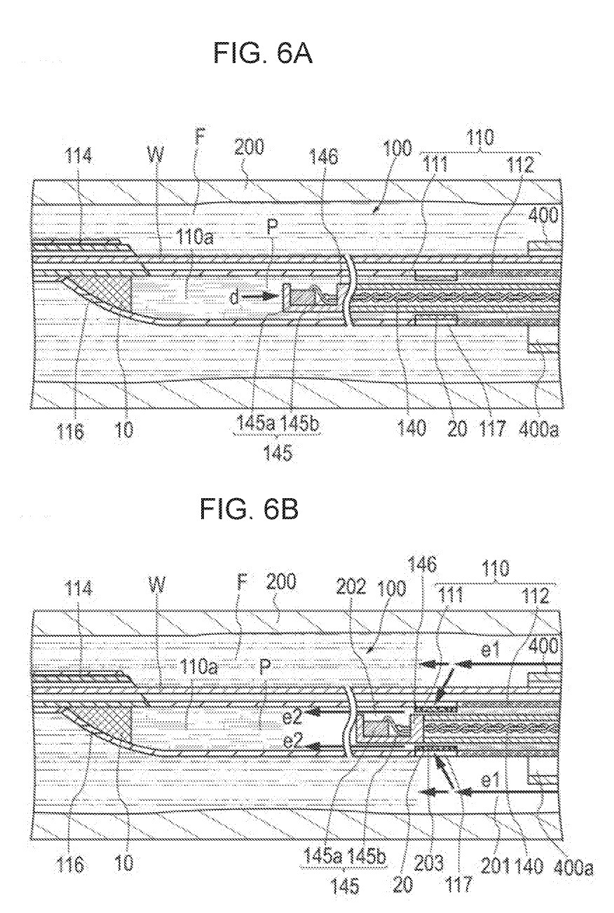

FIG. 1 is a plan view illustrating a state where an external device 300 is connected to a catheter 100 for image diagnosis according to an exemplary embodiment of the disclosure, FIGS. 2A and 2B are diagrams schematically illustrating an overall configuration of the catheter 100 for image diagnosis according to the exemplary embodiment of the disclosure, FIGS. 3 and 4 are diagrams illustrating a configuration of each portion of the catheter 100 for image diagnosis according to the exemplary embodiment of the disclosure, and FIGS. 5A-6B are diagrams used for describing an action of the catheter 100 for image diagnosis.

The catheter 100 for image diagnosis according to the exemplary embodiment is a dual-type catheter that has both intravascular ultrasound (IVUS) and optical coherence tomography (OCT) functions and is capable of switching between the functions or using the functions at the same time. The catheter 100 for image diagnosis is driven by being connected to the external device 300 as illustrated in FIG. 1.

The catheter 100 for image diagnosis will be described with reference to FIGS. 1 to 4.

In summary, the catheter 100 for image diagnosis has a sheath 110 inserted into a body-cavity of a living body, an outer tube 120 disposed on the proximal side of the sheath 110, an inner shaft 130 inserted into the outer tube 120 to be capable of moving forward and backward, a drive shaft 140 that has a distal portion where a signal transferring and receiving unit 145 transferring and receiving a signal is placed, a unit connector 150 disposed on the proximal side of the outer tube 120 and configured to contain the inner shaft 130, and a hub 160 disposed on the proximal side of the inner shaft 130 as illustrated in FIGS. 1, 2(A), and 2(B).

In the description of the specification, the side of the catheter 100 for image diagnosis that is inserted into the body-cavity will be referred to as a distal end or a distal side, the hub 160 side disposed on the catheter 100 for image diagnosis will be referred to as a proximal end or a proximal side, and the extending direction of the sheath 110 will be referred to as an axial direction.

As illustrated in FIG. 2(A), the drive shaft 140 extends up to the inside of the hub 160 through the sheath 110, the outer tube 120 connected to the proximal end of the sheath 110, and the inner shaft 130 inserted into the outer tube 120.

The hub 160, the inner shaft 130, the drive shaft 140, and the signal transferring and receiving unit 145 are interconnected such that each of the hub 160, the inner shaft 130, the drive shaft 140, and the signal transferring and receiving unit 145 integrally moves forward and backward in the axial direction. Accordingly, when an operation for pushing the hub 160 toward the distal side is performed, for example, the inner shaft 130 connected to the hub 160 is pushed into the outer tube 120 and the unit connector 150 and the drive shaft 140 and the signal transferring and receiving unit 145 move to the distal side through the inside of the sheath 110. When an operation for pulling the hub 160 to the proximal side is performed, for example, the inner shaft 130 is pulled out from the outer tube 120 and the unit connector 150 as indicated by an arrow a1 in FIGS. 1 and 2(B) and the drive shaft 140 and the signal transferring and receiving unit 145 move to the proximal side through the inside of the sheath 110 as indicated by an arrow a2.

As illustrated in FIG. 2(A), the distal portion of the inner shaft 130 reaches the vicinity of a relay connector 170 when the inner shaft 130 is pushed to the distal side to the maximum extent possible. At this time, the signal transferring and receiving unit 145 is positioned in the vicinity of the distal side of the sheath 110. The relay connector 170 is a connector connecting the sheath 110 and the outer tube 120 to each other.

As illustrated in FIG. 2(B), a connector 131 for escape prevention is disposed at the distal side of the inner shaft 130. The connector 131 for escape prevention prevents the inner shaft 130 from escaping from the outer tube 120. The connector 131 for escape prevention is configured to be caught at a predetermined position of the inner wall of the unit connector 150 when the hub 160 is pulled to the proximal side to the maximum extent possible, that is, when the inner shaft 130 is pulled out from the outer tube 120 and the unit connector 150 to the maximum extent possible. Note that the disclosure here is not limited to the configuration provided with the connector 131 for escape prevention insofar as escaping of the inner shaft 130 from the outer tube 120 can be prevented. For example, escaping of the inner shaft 130 from the outer tube 120 may also be prevented by parts engaged with each other being disposed at the distal end of the inner shaft 130 and the proximal end of the outer tube 120.

As illustrated in FIG. 3, the drive shaft 140 is provided with a flexible pipe body 140a and an electric signal cable 140b and an optical fiber cable 140c connected to the signal transferring and receiving unit 145 are arranged inside the drive shaft 140. The pipe body 140a can be composed of, for example, multiple layers of coils that have different winding directions around an axis. Examples of the material of the coils include stainless steel and a nickel-titanium (Ni—Ti) alloy. The electric signal cable 140b can be composed of, for example, a twisted pair cable and a coaxial cable.

The signal transferring and receiving unit 145 has an ultrasound transferring and receiving unit 145a transferring and receiving an ultrasound wave and an optical transceiver 145b transferring and receiving light.

The ultrasound transferring and receiving unit 145a is provided with an oscillator, transfers an ultrasound wave based on a pulse signal into the body-cavity, and receives the ultrasound wave reflected from the body-cavity. The ultrasound transferring and receiving unit 145a is electrically connected to an electrode terminal 166 (refer to FIG. 4) via the electric signal cable 140b. A piezoelectric material such as ceramics and a crystal can be used as the oscillator of the ultrasound transferring and receiving unit 145a.

The optical transceiver 145b continuously transfers transferred measurement light into the body-cavity and continuously receives reflected light from a biological tissue in the body-cavity. The optical transceiver 145b has a ball lens (optical element) disposed at the distal end of the optical fiber cable 140c and provided with a lens function for collecting light and a reflection function for reflecting light.

The signal transferring and receiving unit 145 is accommodated in a housing 146. The proximal side of the housing 146 is connected to the drive shaft 140. The housing 146 is shaped such that an opening portion is disposed in a cylindrical surface of a cylindrical metal pipe and is formed by metal ingot shaving, metal powder injection molding (MIM), or the like.

As illustrated in FIG. 3, the sheath 110 is provided with a lumen 110a into which the drive shaft 140 is inserted to be capable of moving forward and backward. A guide wire insertion member 114 provided with a guide wire lumen 114a is attached to the distal portion of the sheath 110, the guide wire lumen 114a is disposed in parallel to the lumen 110a disposed in the sheath 110, and a guide wire W can be inserted into the guide wire lumen 114a. The sheath 110 and the guide wire insertion member 114 can be integrally configured by heat-welding or the like. A marker 115 that has X-ray contrast is disposed in the guide wire insertion member 114. The marker 115 is composed of a metal coil with high radiopacity such as Pt, Au, and Ir.

The distal portion of the sheath 110, which is the range in which the signal transferring and receiving unit 145 moves in the axial direction of the sheath 110, constitutes a window portion 111 formed to have a higher level of permeability of an inspection wave such as an ultrasound wave and light than the other sites.

The window portion 111 and the guide wire insertion member 114 of the sheath 110 are formed of a flexible material, the material is not particularly limited, examples of the material include various thermoplastic elastomers such as polyethylene, styrene, polyolefin, polyurethane, polyester, polyamide, polyimide, polybutadiene, trans-polyisoprene, fluororubber, and chlorinated polyethylene, and a laminate, a polymer blend, a polymer alloy, or the like in which one or at least two of the above are combined can also be used. Note that a hydrophilic lubricating coating layer exhibiting lubricity when wet can be placed on the outer surface of the sheath 110.

The side of the sheath 110 that is closer to the proximal side than the window portion 111 has a reinforced portion 112 reinforced by a material more rigid than the window portion 111. The reinforced portion 112 is formed by, for example, a reinforcement body 112r being arranged in which a metal wire such as a stainless steel wire is braided in a mesh shape in a flexible tubular member such as resin. The tubular member is formed of the same material as the window portion 111.

As illustrated in FIG. 3, a first communication hole 116 for communication between the inside and the outside of the lumen 110a is formed in the distal portion of the window portion 111 of the sheath 110. In addition, a second communication hole 117 for communication between the inside and the outside of the lumen 110a is formed between the window portion 111 and the reinforced portion 112.

The sheath 110 further has a first filter (corresponding to a first restriction unit) 10 disposed to cover the first communication hole 116 and a second filter (corresponding to a second restriction unit) 20 disposed to cover the second communication hole 117 over the entire circumference in the circumferential direction of the sheath 110.

The first filter 10 is composed of a component placed in the first communication hole 116, allowing gas circulation, and scattering or absorbing light contained in blood 201, examples of which include a known porous filter restricting erythrocyte circulation. The porous filter can be composed of a known gas-liquid separation filter that has multiple through-holes, allows gas circulation, and restricts liquid circulation. In the exemplary embodiment, the porous filter restricts circulation of a priming solution P as a liquid and the blood 201 (corresponding to a body fluid). Note that the blood 201 contains a liquid component 202 and a component 203 scattering or absorbing light.

Polyethylene, polypropylene, polystyrene, polyimide, and so on can be used as the material of the first filter 10. Preferably, a highly waterproof fluororesin can be used as the material of the first filter 10. More preferably, polytetrafluoroethylene (PTFE) can be used as the material of the first filter 10. Preferably, the diameter of the through-hole formed in the porous filter constituting the first filter 10 ranges from 0.01 to 0.1 micrometer.

The second filter 20 is composed of a component placed in the second communication hole 117, allowing circulation of gas, the priming solution P, and the liquid component 202 contained in the blood 201 such as blood plasma, and scattering or absorbing the light contained in the blood 201, examples of which include a porous filter restricting erythrocyte circulation. The porous filter can be composed of a known blood plasma separation filter that has multiple through-holes, allows circulation of the blood plasma 202, the priming solution P, and gas, and restricts circulation of cellular components such as the erythrocyte 203 and a platelet by capturing the cellular components.

Cellulose-mixed ester, polyvinylidene difluoride, polytetrafluoroethylene, polycarbonate, polypropylene, polyester, nylon, glass, alumina, and so on can be used as the material of the second filter 20. Preferably, the diameter of the through-hole formed in the porous filter constituting the second filter 20 ranges from 0.05 to 2.0 micrometers. The protein, lipid, and the like in the blood plasma 202 may be clogged up when the diameter is less than 0.05 micrometers, and the erythrocyte 203 may pass through the through-hole due to the deformability of the erythrocyte 203 when the diameter exceeds 2.0 micrometers. More preferably, the through-hole has a diameter of 0.1 to 1.5 micrometers.

As illustrated in FIG. 4, the hub 160 has a hub main body 161 that has a hollow shape, a port 162 communicating with the inside of the hub main body 161, projections 163a and 163b for direction confirmation for confirming the direction of the hub 160 during connection to the external device 300, a sealing member 164a sealing the side of the hub 160 that is closer to the proximal side than the port 162, a connection pipe 164b holding the drive shaft 140, a bearing 164c rotatably supporting the connection pipe 164b, and a connector unit 165 in which the electrode terminal 166 mechanically and electrically connected to the external device 300 is placed.

The inner shaft 130 is connected to the distal portion of the hub main body 161. The drive shaft 140 is pulled out from the inner shaft 130 inside the hub main body 161. A protective pipe 133 is placed between the inner shaft 130 and the drive shaft 140. The protective pipe 133 suppresses the vibration of the drive shaft 140 that is generated during a pull-back operation.

The connection pipe 164b holds the drive shaft 140 at the distal end of the connection pipe 164b, which is the end portion on the side opposite to a rotor 167, in order to transfer rotation of the rotor 167 to the drive shaft 140. The electric signal cable 140b and the optical fiber cable 140c (refer to FIG. 4) are inserted into the connection pipe 164b, one end of the electric signal cable 140b and one end of the optical fiber cable 140c are connected to the electrode terminal 166, and the other ends of the electric signal cable 140b and the optical fiber cable 140c are respectively connected to the ultrasound transferring and receiving unit 145a and the optical transceiver 145b through the inside of the drive shaft 140. A reception signal of the ultrasound transferring and receiving unit 145a and the optical transceiver 145b is transferred to the external device 300 via the electrode terminal 166 and displayed as an image after a predetermined treatment is performed.

Referring back to FIG. 1, the catheter 100 for image diagnosis is driven by being connected to the external device 300. As described above, the external device 300 is connected to the connector unit 165 disposed on the proximal side of the hub 160.

In addition, the external device 300 has a motor 300a as a power source for rotating the drive shaft 140 and a motor 300b as a power source for moving the drive shaft 140 in the axial direction. The rotational motion of the motor 300b is converted into an axial-direction motion by a ball screw element 300c connected to the motor 300b.

The operation of the external device 300 is controlled by a control apparatus 320 electrically connected to the external device 300. The control apparatus 320 includes a central processing unit (CPU) and a memory as its main components. The control apparatus 320 is electrically connected to a monitor 330.

An example of use of the catheter 100 for image diagnosis will be described below.

Firstly, the external device 300 is connected to the connector unit 165 of the catheter 100 for image diagnosis as illustrated in FIG. 1. A user connects a syringe S containing the priming solution P such as a saline solution to the port 162. The user injects the priming solution P into the lumen 110a of the sheath 110 as illustrated in FIG. 5(A) by pressing the plunger of the syringe S.

Once the priming solution P is injected into the lumen 110a of the catheter 100 for image diagnosis, the air in the lumen 110a is extruded to the distal side by the pressure of the priming solution P. Since the first filter 10 allows gas circulation, the air is discharged to the outside of the lumen 110a from the first communication hole 116 via the first filter 10 as indicated by an arrow b1 in FIG. 5(A). Meanwhile, the first filter 10 restricts the circulation of the priming solution P, and thus the priming solution P remains in the lumen 110a. As a result, the air in the lumen 110a can be smoothly discharged and unnecessary discharge of the priming solution P can be prevented during a priming treatment, and thus the priming treatment can be completed within a shorter period of time. In addition, since the second filter 20 allows the passage of gas and the priming solution P, the air and the priming solution P are discharged to the outside of the lumen 110a from the second communication hole 117 via the second filter 20 as indicated by an arrow b2 in FIG. 5(A). The resistance during the passage of the priming solution P through the second filter 20 exceeds the resistance during the passage of the air through the second filter 20 because of surface tension. Accordingly, in the early stage following the initiation of the priming treatment, only the air is discharged from the second communication hole 117. Subsequently, the air is discharged to the outside from the inside of the lumen 110a, and the injection pressure of the priming solution P sharply increases when the inside of the lumen 110a is replaced by the priming solution P up to the distal end. Completion of the priming treatment can be confirmed from the increase in injection pressure.

Air may remain in the lumen 110a during the priming treatment in, for example, the drive shaft 140 and the hub 160 on the hand-side. A re-priming treatment may be performed for extruding the air to the outside of the lumen 110a by performing the priming treatment again so that the air does not move to the distal side in the lumen 110a. The air passes around the signal transferring and receiving unit 145 in the window portion 111 when the air is allowed to be discharged via the first communication hole 116 during the re-priming treatment, and thus the air may remain on the surface of the signal transferring and receiving unit 145. An unclear diagnostic image may result from the presence of the air around the signal transferring and receiving unit 145. In the exemplary embodiment, air and the priming solution P are discharged to the outside of the lumen 110a from the second communication hole 117 via the second filter 20 as described above. Since the air can be discharged during the re-priming treatment from the second communication hole 117, which is placed closer to the proximal side than the window portion 111, the air remaining around the signal transferring and receiving unit 145 after moving to the distal side and passing through the window portion 111 can be inhibited. In addition, since the first communication hole 116 allows only the air to pass therethrough and the priming solution P is capable of passing through the second communication hole 117 via the second filter 20, a flow of the priming solution P discharged from the second communication hole 117 to the outside of the lumen 110a is generated. In accordance with this flow, the air can be discharged to the outside of the lumen 110a without passing through the window portion 111.

After the priming treatment, the user moves the signal transferring and receiving unit 145 to the distal side by pushing the hub 160 until the hub 160 is attached to the proximal end of the unit connector 150 as illustrated in FIG. 2(A). In this state, the catheter 100 for image diagnosis is inserted into a lumen 400a of a guiding catheter 400. Note that the guiding catheter 400 is inserted in advance into a blood vessel 200 along the guide wire W. Then, the catheter 100 for image diagnosis is advanced along the lumen 400a and protrudes from the distal side opening portion of the guiding catheter 400. Subsequently, the catheter 100 for image diagnosis is further pushed forward along the guide wire W while the guide wire W is inserted into the guide wire lumen 114a as illustrated in FIG. 5(B), and then the catheter 100 for image diagnosis is inserted into a target position in the blood vessel 200. Note that a known guiding catheter that is provided with a port (not illustrated) to which a syringe (not illustrated) can be connected in its proximal portion can be used as the guiding catheter 400.

Subsequently, a flush treatment is performed so that the blood 201 in the blood vessel 200 is washed away with a flush solution F such as a contrast agent. As in the priming treatment described above, a syringe containing the flush solution F is connected to a port of the guiding catheter 400 and the flush solution F is injected into the lumen 400a of the guiding catheter 400 by the plunger of the syringe being pressed. As indicated by an arrow c in FIG. 5(C), the flush solution F is introduced into the blood vessel 200 through the lumen 400a of the guiding catheter 400 and via the distal side opening portion thereof. The blood 201 around the window portion 111 of the sheath 110 is swept away by the introduced flush solution F and a state occurs where the space around the window portion 111 is filled with the flush solution F.

When a tomographic image is obtained at the target position in the blood vessel 200, the signal transferring and receiving unit 145 transfers and receives the inspection wave while moving to the proximal side with the drive shaft 140 as indicated by an arrow d in FIG. 6(A). At this time, the signal transferring and receiving unit 145 rotates with the drive shaft 140.

Once the drive shaft 140 is moved to the proximal side, the internal pressure of the distal part of the lumen 110a decreases. The internal pressure of this part is lower than that of the outside of the lumen 110a and is a negative pressure. In addition, once some time elapses after the flush treatment is performed, the blood 201 flows in from the proximal side as indicated by an arrow e1 in FIG. 6(B) due to the blood flow in the blood vessel 200. The blood 201 that flows in is guided to the second communication hole 117 communicating with the outside of the lumen 110a due to the negative pressure in the lumen 110a. At this time, the blood plasma 202 flows into the lumen 110a from the outside of the lumen 110a via the second communication hole 117 as indicated by an arrow e2 in FIG. 6(B) since the second filter 20 allows the circulation of the blood plasma 202 contained in the blood 201. As a result, the negative pressure in the lumen 110a can be eliminated. Furthermore, the amount of the blood 201 that flows into the side closer to the distal side than the second communication hole 117 can be decreased by the blood 201 flowing in from the proximal side in the blood vessel 200 being guided to the second communication hole 117. Accordingly, the region where the blood 201 is swept away by the flush treatment can be narrowed. As a result, the burden on a patient can be reduced by the amount of the flush solution F used for the flush treatment being reduced.

Moreover, the second filter 20 restricts the circulation of the erythrocyte 203 by capturing the erythrocyte 203 contained in the blood 201. As a result, a flow of the erythrocyte 203 contained in the blood 201 into the lumen 110a can be restricted, and thus a diagnostic image becoming unclear due to the erythrocyte 203 can be inhibited. In addition, the negative pressure in the lumen 110a can be eliminated since the first filter 10 restricts a flow of the blood plasma 202 flowing in from the second communication hole 117 during the pull-back operation to the outside of the lumen 110a via the first communication hole 116.

The control apparatus 320 controls the motor 300a illustrated in FIG. 1 and controls the rotation of the drive shaft 140 around an axis. In addition, the control apparatus 320 controls the motor 300b and controls an axial-direction movement of the drive shaft 140.

The signal transferring and receiving unit 145 transfers an ultrasound wave and light into the body based on a signal sent from the control apparatus 320. The signal that corresponds to the reflected wave and the reflected light which are received by the signal transferring and receiving unit 145 is sent to the control apparatus 320 via the drive shaft 140 and the external device 300. The control apparatus 320 generates the tomographic image of the body-cavity based on the signal sent from the signal transferring and receiving unit 145 and displays the generated image on the monitor 330.

The connector unit 165 disposed in the hub 160 rotates in a state of being connected to the external device 300, and the drive shaft 140 rotates in conjunction with the rotation. The connector unit 165 and the drive shaft 140 have a rotational speed of, for example, 1,800 rpm.

As described above, the sheath 110 of the catheter 100 for image diagnosis according to the exemplary embodiment has the first filter 10 and the second filter 20, the first filter 10 is placed in the first communication hole 116, allows a gas flow to the outside from the inside of the lumen 110a via the first communication hole 116, and restricts a flow of the erythrocyte 203 to the inside from the outside of the lumen 110a, and the second filter 20 is placed in the second communication hole 117, allows a flow of the blood plasma 202 contained in the blood 201 to the inside from the outside of the lumen 110a via the second communication hole 117, and restricts a flow of the erythrocyte 203 to the inside from the outside of the lumen 110a.

With the catheter 100 for image diagnosis configured as described above, the air in the lumen 110a can be discharged from the first communication hole 116 and the second communication hole 117 during the priming treatment, and thus the air in the lumen 110a can be replaced with the priming solution P up to the distal end. In addition, during the pull-back operation, the first filter 10 and the second filter 20 are capable of restricting a flow of the erythrocyte 203 contained in the blood 201 to the inside from the outside of the lumen 110a via the first communication hole 116 and the second communication hole 117. As a result, a diagnostic image becoming unclear due to the erythrocyte 203 can be inhibited. Furthermore, the negative pressure in the lumen 110a can be eliminated by the blood plasma 202 contained in the blood 201 flowing in from the proximal side in the blood vessel 200 flowing into the lumen 110a via the second communication hole 117. Moreover, the amount of the blood 201 that flows into the side which is closer to the distal side than the second communication hole 117 can be decreased by the blood 201 flowing in from the proximal side in the blood vessel 200 being guided to the second communication hole 117. As a result, the region where the blood 201 is swept away by the flush treatment can be narrowed, and thus the amount of the flush solution F used for the flush treatment can be reduced and the burden on a patient can be reduced. In addition, air can be discharged during the re-priming treatment from the second communication hole 117 located closer to the proximal side than the window portion 111, and thus air remaining around the signal transferring and receiving unit 145 after moving to the distal side can be inhibited.

Moreover, the first filter 10 further restricts a flow of the blood plasma 202 contained in the blood 201 and the priming solution P to the outside from the inside of the lumen 110a. As a result, the air in the lumen 110a can be smoothly discharged and unnecessary discharge of the priming solution P can be prevented during the priming treatment, and thus the priming treatment can be efficiently performed. The first filter 10 restricts a flow of the blood plasma 202 flowing in from the second communication hole 117 to the outside of the lumen 110a via the first communication hole 116, and thus the negative pressure in the lumen 110a that is generated during the pull-back operation can be eliminated. Since the first communication hole 116 allows only the air to pass therethrough and the second communication hole 117 allows the priming solution P to pass through the second filter 20 during the re-priming treatment, a flow discharged from the second communication hole 117 to the outside of the lumen 110a is generated. In accordance with this flow, the air can be discharged to the outside of the lumen 110a without passing through the window portion 111. As a result, air moving to the distal side and passing and remaining around the signal transferring and receiving unit 145 can be inhibited.

The second filter 20 is formed over the entire circumference in the circumferential direction of the sheath 110, and thus the surface area of the second filter 20 facing the blood 201 around the second communication hole 117 increases. Accordingly, the erythrocyte 203 can be captured more efficiently, and the negative pressure in the lumen 110a can be eliminated more reliably by the amount of flow of the blood plasma 202 to the inside from the outside of the lumen 110a being increased.

The sheath 110 has the window portion 111 transferring an ultrasound wave and light in the distal portion, and the second filter 20 is placed on the side that is closer to the proximal side than the window portion 111. The blood 201 flowing in from the proximal side during the pull-back operation is guided to the second communication hole 117 where the second filter 20 is placed by the negative pressure in the lumen 110a, and thus a flow of the blood 201 into the side that is closer to the distal side than the second communication hole 117 can be inhibited. Since the second filter 20 is placed on the side that is closer to the proximal side than the window portion 111, an inflow of the blood 201 around the window portion 111, which is the range of movement of the signal transferring and receiving unit 145, can be inhibited, and thus a diagnostic image becoming unclear due to the erythrocyte 203 contained in the blood 201 can be inhibited more reliably.

The first filter 10 is formed from a porous filter disposed to cover the first communication hole 116, and the second filter 20 is formed from a porous filter disposed to cover the second communication hole 117. As a result, a porous filter that is widely used can be used, and thus manufacturing costs can be reduced.

Although the catheter for image diagnosis according to the disclosure herein has been described by way of an exemplary embodiment, the disclosure is not limited to the configuration described in the exemplary embodiment and can be appropriately changed based on the description of the claims.

For example, although the first filter in the exemplary embodiment of the disclosure restricts blood and priming solution circulation, the disclosure is not limited thereto and the first filter may be formed of the same material as the second filter insofar as the first filter is capable of restricting an inflow of a component scattering or absorbing light contained in a body fluid. In this manner, the priming solution can be discharged via the first communication hole during the priming treatment. In addition, a diagnostic image becoming unclear can be inhibited by the inflow of the component scattering or absorbing light being restricted.

The body fluid to which the disclosure is applied is not limited to blood, and the disclosure can also be applied to urine or the like. In this case, examples of the component scattering or absorbing light include the protein that is contained in the urine.

Although the second filter in the exemplary embodiment is formed over the entire circumference in the circumferential direction of the sheath, the disclosure is not limited thereto and the second filter may also be formed at a part in the circumferential direction of the sheath.

Although the first communication hole is disposed in the distal surface of the sheath, the first communication hole may also be disposed in a side surface on the side to which the guide wire insertion member of the sheath is attached.

Although the first filter 10 and the second filter 20 are composed of porous filters, the disclosure is not limited thereto and a filter function may also be provided by a nonwoven fabric, a chemically treated membrane, or the like in an alternative configuration.

The detailed description above describes features, characteristics and operational aspects of embodiments of a catheter for image diagnosis disclosed herein. The disclosure and the present invention are not limited, however, to the precise embodiments and variations described. Various changes, modifications and equivalents could be effected by one skilled in the art without departing from the spirit and scope of the disclosure as defined in the appended claims. It is expressly intended that all such changes, modifications and equivalents which fall within the scope of the claims are embraced by the claims.

What is claimed is:

1. A catheter for image diagnosis comprising:
a drive shaft including a distal portion having an ultrasound transferring and receiving unit transferring and receiving an ultrasound wave and an optical transceiver transferring and receiving light; and
a sheath including a lumen configured for the drive shaft to be inserted therein such that the drive shaft is movable in a forward and a backward direction,
wherein the sheath includes:
a first communication hole in a distal portion and formed such that an inside and an outside of the lumen communicate with each other;
a second communication hole on a side closer to a proximal side than the first communication hole and formed such that the inside and the outside of the lumen communicate with each other;
a first restriction unit placed in the first communication hole, allowing a flow of gas to the outside from the inside of the lumen via the first communication hole, and restricting a flow of a component scattering or absorbing light contained in a body fluid to the inside from the outside of the lumen; and
a second restriction unit placed in the second communication hole, allowing a flow of a liquid component contained in the body fluid to the inside from the outside of the lumen via the second communication hole, and restricting a flow of the component scattering or absorbing light contained in the body fluid to the inside from the outside of the lumen.

2. The catheter for image diagnosis according to claim 1,
wherein the first restriction unit further restricts a flow of the liquid component contained in the body fluid and a priming solution to the outside from the inside of the lumen.

3. The catheter for image diagnosis according to claim 1,
wherein the second restriction unit is formed over an entire circumference in a circumferential direction of the sheath.

4. The catheter for image diagnosis according to claim 1,
wherein the sheath has a window portion transferring an ultrasound wave and light in a distal portion, and
wherein the second restriction unit is placed on a side closer to the proximal side than the window portion.

5. The catheter for image diagnosis according to claim 1,
wherein the first restriction unit is formed of a porous filter disposed to cover the first communication hole, and
wherein the second restriction unit is formed of a porous filter disposed to cover the second communication hole.

6. A catheter comprising:
a sheath including a lumen;
a first communication hole in a distal portion of the sheath configured for communication between an inside and an outside of the lumen;
a second communication hole proximal to the first communication hole and configured for communication between the inside and the outside of the lumen;
a first restriction unit placed in the first communication hole, allowing a flow of gas to the outside from the inside of the lumen via the first communication hole, and restricting a flow of a first bodily fluid component from the outside to the inside of the lumen; and
a second restriction unit placed in the second communication hole, allowing a flow of a second bodily fluid component to the inside from the outside of the lumen via the second communication hole, and restricting a flow of the first bodily fluid component from the outside to the inside of the lumen.

7. The catheter according to claim 6, wherein the first restriction unit further restricts a flow of the second bodily fluid component from the inside to the outside of the lumen.

8. The catheter according to claim 7, wherein the first restriction unit further restricts a flow of a priming solution from the inside to the outside of the lumen.

9. The catheter according to claim 6,
wherein the second restriction unit is formed over at least a portion of a circumference of the sheath in a circumferential direction.

10. The catheter according to claim 6,
wherein the sheath includes a window portion, and the second restriction unit is disposed proximal to the window portion.

11. The catheter according to claim 10,
wherein the sheath includes a reinforced portion on a portion proximal to the window portion, the reinforced portion being formed from a material more rigid that a material forming the window portion.

12. The catheter according to claim 6,
wherein the first restriction unit is formed of a porous filter disposed to cover the first communication hole.

13. The catheter according to claim 12,
wherein the second restriction unit is formed of a porous filter disposed to cover the second communication hole.

14. The catheter according to claim 6,
further comprising a drive shaft including a distal portion having an ultrasound transferring and receiving unit transferring and receiving an ultrasound wave and an optical transceiver transferring and receiving light, the drive shaft configured to be movable in the lumen of the sheath in a forward and backward direction.

15. A method of using a catheter for image diagnosis comprising:
priming the catheter by:
injecting a priming solution into a lumen of a sheath of the catheter;
discharging air to outside of the lumen of the sheath through a first communication hole have a first filter;
restricting discharge of the priming solution through the first communication hole with the first filter; and
discharging air and the priming solution to outside of the lumen of the sheath through a second communication hole having a second filter;
advancing the catheter through a guiding catheter to a target position in a blood vessel;
flushing the guiding catheter by injecting a flush solution into a lumen of the guiding catheter;
obtaining a tomographic image at the target position in the blood vessel while moving a drive shaft including a signal transferring and receiving unit in a proximal direction within the sheath of the catheter;
developing a negative pressure in the lumen of the sheath and causing blood to flow into the guiding catheter,
allowing a second component of blood to flow to inside of the lumen of the sheath through the second communication hole having the second filter;
restricting a flow of the second component of blood flowing in to inside of the lumen through the second communication hole; and
restricting a first component of blood from flowing to inside of the lumen of the sheath through the second communication hole having the second filter.

16. The method of claim 15, wherein restricting the flow of the second component of blood flowing in to inside of the lumen through the second communication hole includes restricting discharge of the second component to outside of the lumen through the first communication hole with the first filter.

17. The method of claim 15, further comprising repriming the catheter.
